(19) United States
(12) Patent Application Publication
(10) Pub. No.: US 2025/0246312 A1
(43) Pub. Date: Jul. 31, 2025

(54) SYSTEMS AND METHODS FOR DETERMINING A DEPRESSION SCORE AND/OR RELAPSE

(71) Applicant: Dalia Care SAS, Paris (FR)

(72) Inventors: Nour Hakiki BELHADI, Paris (FR); Thibaut BARROYER, Paris (FR); Christian FRISCH, Paris (FR)

(73) Assignee: Dalia Care SAS, Paris (FR)

(21) Appl. No.: 19/035,652

(22) Filed: Jan. 23, 2025

(30) Foreign Application Priority Data
Jan. 25, 2024 (EP) 24305135.6

(51) Int. Cl. G16H 50/30; G16H 10/20; G16H 10/60; G16H 40/67

(52) U.S. Cl. CPC G16H 50/30; G16H 10/20; G16H 10/60; G16H 40/67

(57) ABSTRACT

Systems and methods are provided herein for monitoring mental health and/or detecting and/or predicting a depressive relapse and/or determining a depressive state, label, and/or score using one or more machine learning models with one or more neural networks. Machine learning models may be trained using general data not specific to a certain user then may be tailored to a specific user and calibrated using user data that is associated with a time period as well as survey or other assessment data also associated with that time period. Newly generated user data may then be processed by the trained machine learning algorithm which may generate one or more score indicative a risk of a depression relapse and/or a depressive state and/or label. Based on the score, alerts, reports, and/or recommendations of actions for the user to take may be generated.

### CROSS-REFERENCE TO RELATED APPLICATIONS

This application claims priority to European Application No. 24305135.6, filed on Jan. 25, 2024, the entire contents of which are incorporated herein by reference.

### TECHNICAL FIELD

This technology relates, in general, to a system having artificial intelligence and machine learning functionality for detecting and/or predicting depressive relapse and/or a depressive state.

### BACKGROUND

According to the National Institute of Mental Health (NIMH), an estimated 23.1% of adults in the United States (59.3 million people) live with a mental illness. In the United States alone, it is estimated that more than 8% of the population, about 21 million people, suffer from depression, a form of mental illness, and about 15% of the population ages 12-27 (around 3.7 million people), are affected by depression. Similarly, in France around 12.5% of the population experiences a depressive episode each year. For those who experience depressive episode, around 80% experience a depressive episode relapse.

Unfortunately, for many, mental illness goes unidentified and untreated. In the United States, there is only 1 psychiatrist per 1968 depressed patients. In France this ratio is only slightly better with only 1 psychiatrist per 560 depressed patients. In addition to limited number of trained healthcare providers, those suffering from mental illness may lack the funds and/or insurance required to meet regularly with a healthcare provider, may be located a significant distance from the nearest healthcare provider, and/or may lack the funds and/or insurance for appropriate treatment (e.g., medication).

While early detection of the signs of depressive relapse may improve quality of treatment and prevent or reduce the severity and/or duration of a relapse, it may be cost prohibitive and otherwise logistically not feasible to continuously monitor an individual's behavior and mental state. Even for individuals that periodically visit with healthcare providers, such patients may stop seeing their healthcare provider once their mental health is stabilized and/or may not recognize early signs of depression relapse or may otherwise feel ashamed and may not seek help. As time passes, the severity of depression may increase and may become harder to treat and may last longer.

Accordingly, there is a need for improved methods and systems for monitoring mental health of a patient and analyzing and/or processing various user data indicative of a mental state of an individual and detecting and/or predicting depressive relapse.

### SUMMARY

Provided herein are systems for monitoring mental health and/or detecting and/or predicting a depressive relapse and/or depressive state, label, and/or score using one or more models with one or more neural networks. Machine learning models may be trained using general data not specific to a certain user then may be tailored to a specific user and calibrated using user data such as physiological, behavioral, and/or survey data, for example, that is associated with a time period also associated with survey or other assessment data indicating the user's mental health state at that time period. Newly generated user data may then be processed by the trained machine learning algorithm which may generate one or more score indicative a risk of a depression relapse and/or a depressive state and/or label. Based on the score, alerts, reports, and/or recommendations of actions for the user to take may be generated.

A method is provided herein for monitoring mental health of a user. The method may include training a machine learning algorithm using a plurality of general user data including o general physiological, general activity and/or general sleep data corresponding to a plurality of general users different than the user and generate a score indicative of a degree of depression, receiving a plurality of first user data from at least one user device, the plurality of first user data including first physiological, first activity, and/or first sleep data corresponding to the user and associated with a first time period, receiving survey data corresponding to at least one depressive state of the user and associated with the first time period, updating the machine learning algorithm by further training the machine learning algorithm using the plurality of first user data and the survey data resulting in an updated machine learning algorithm, receiving a plurality of second user data from at least one user device, the plurality of second user data corresponding to second physiological, second activity, and/or second sleep data corresponding to the user and associated with a second time period after the first time period, transforming the plurality of second user data into at least one vector, the at least one vector representative of the plurality of second user data, and generating a first score using the updated machine learning algorithm and the at least one vector, the first score indicative of a first degree of depression of the user at the second time period.

The updated machine learning algorithm may include an input layer, a plurality of feature extraction layers, a plurality of recurrent layers, and at least one dense layer that is a fully connected layer. The method may include determining historical data corresponding to the user and including demographic data, medical history data, clinical data, and/or family history data. The at least the dense layer may processes the historical data and the updated machine learning algorithm generates the first score based on both the historical data and the plurality of second user data. The plurality of second user data may include first data corresponding to a first 24 hour period within the second time period and second data corresponding to a second 24 hour period within the second time period, wherein the plurality of feature extraction layers may include a first feature extraction layer and a second feature extraction layer, and wherein the first feature extraction layer processes the first data and the second feature extraction layer processes the second data.

The updated machine learning algorithm may be a recurrent deep neural network (RDNN). Alternatively, the updated machine learning algorithm may include a regression model and/or a classification model. The method may include comparing the first score to a predefined threshold value corresponding to a depression relapse; and determining the first score exceeds the predefined threshold value indicating a presence of the depression relapse at the second time period. The method may further include sending, based on the first score exceeding the predefined threshold value, an alert to a user device and/or a health care provider device corresponding to the presence of the depression relapse at the second time period. The alert may be sent to the user device and the method may further include receiving feedback data from the user device after the alert is sent to the user device. The feedback data may reject the first score and/or the alert.

The method may further include updating the updated machine learning algorithm by further training the updated machine learning algorithm using the feedback data. The method may further include determining, based on the first score exceeding the predefined threshold value, a corrective action corresponding to the presence of the depression relapse, and sending instructions corresponding to the corrective action to the user device. The method of corrective action may correspond to one of an exercise recommendation, a diet recommendation, a medication recommendation, or a recommendation to meet with a healthcare provider. The method may include generating, automatically, a report including the plurality of second user data and the first score, and sending the report to a healthcare provider device. The plurality of user data may include heart rate data, electrocardiogram data, sleep data, temperature data, breathing data, step data, exercise data, screen time data, media data, call data, text data, email data, and/or location data.

A system is provided herein for monitoring mental health of a user. The system may include memory configured to store computer-executable instructions, and at least one computer processor configured to access memory and execute the computer-executable instructions to: train a machine learning algorithm using a plurality of general user data including general physiological, general activity and/or general sleep data corresponding to a plurality of general users different than the user and generate a score indicative of a degree of depression, receive a plurality of first user data from at least one user device, the plurality of first user data including first physiological, first activity, and/or first sleep data corresponding to the user and associated with a first time period, receive survey data corresponding to at least one depressive state of the user and associated with the first time period, update the machine learning algorithm by further training the machine learning algorithm using the plurality of first user data and the survey data resulting in an updated machine learning algorithm, receive a plurality of second user data from at least one user device, the plurality of second user data corresponding to second physiological, second activity, and/or second sleep data corresponding to the user and associated with a second time period after the first time period, transform the plurality of second user data into at least one vector, the at least one vector representative of the plurality of second user data, and generate a first score using the updated machine learning algorithm and the at least one vector, the first score indicative of a first degree of depression of the user at the second time period.

The updated machine learning algorithm including an input layer, a plurality of feature extraction layers, a plurality of recurrent layers, and at least one dense layer that is a fully connected layer. The at least one computer processor may be further configured to access memory and execute the computer-executable instructions to determine historical data corresponding to the user including demographic data, medical history data, clinical data, and/or family history data. The at least the dense layer may processes the historical data and the updated machine learning algorithm may generate the first score based on both the historical data and the plurality of second user data.

The plurality of second user data including first data corresponding to a first 24 hour period within the second time period and second data corresponding to a second 24 hour period within the second time period, wherein the plurality of feature extraction layers may include a first feature extraction layer and a second feature extraction layer, and wherein the first feature extraction layer processes the first data and the second feature extraction layer processes the second data. The updated machine learning algorithm may be a recurrent deep neural network (RDNN). The updated machine learning algorithm may include a regression model and a classification model. The at least one computer processor may be further configured to access memory and execute the computer-executable instructions to compare the first score to a predefined threshold value corresponding to a depression relapse, and determine the first score exceeds the predefined threshold value indicating a presence of the depression relapse at the second time period. The at least one computer processor is further configured to access memory and execute the computer-executable instructions to send, based on the first score exceeding the predefined threshold value, an alert to a user device and/or a health care provider device corresponding to the presence of the depression relapse at the second time period.

The alert may be sent to the user device and wherein the at least one computer processor may be further configured to access memory and execute the computer-executable instructions to receive feedback data from the user device after the alert is sent to the user device, the feedback data rejecting the first score and/or the alert. The at least one computer processor is further configured to access memory and execute the computer-executable instructions to update the updated machine learning algorithm by further training the updated machine learning algorithm using the feedback data. The at least one computer processor is further configured to access memory and execute the computer-executable instructions to: determine, based on the first score exceeding the predefined threshold value, a corrective action corresponding to the presence of the depression relapse, and send instructions corresponding to the corrective action to the user device. The corrective action may corresponds to one of an exercise recommendation, a diet recommendation, a medication recommendation, or a recommendation to meet with a healthcare provider. The at least one computer processor may be further configured to access memory and execute the computer-executable instructions to generate, automatically, a report including the plurality of second user data and the first score, and send the report to a healthcare provider device. The plurality of user data may include heart rate data, electrocardiogram data, sleep data, temperature data, breathing data, step data, exercise data, screen time data, media data, call data, text data, email data, and/or location data.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1A illustrates a system for monitoring mental health and/or detecting and/or predicting a depressive relapse.

FIG. 1B illustrates a schematic view of data flow between one or more user devices, a health care provider device, and a back end of mental health monitoring system.

FIG. 2A illustrates a schematic view of a depressive relapse detection and/or prediction model using one or more neural networks.

FIG. 2B illustrates a schematic view of a mental health monitoring model using one or more neural networks.

FIG. 3A illustrates a process flow for training one or more models trained to output a scores and/or labels indicative of a severity of depression.

FIG. 3B illustrates a process flow for detecting and/or predicting a depressive relapse using one or more models trained to output a scores and/or labels indicative of a severity of depression.

FIG. 4 illustrates a plot of calibration and monitoring data as well as threshold values corresponding to a healthy mental state for the patient.

FIGS. 5A-5F are exemplary graphic user interfaces and key points relating to the system for monitoring mental health and/or detecting and/or predicting a depressive relapse.

FIG. 6 is a schematic block diagram of a computing device, in accordance with one or more example embodiments of the disclosure.

The foregoing and other features of the present technology will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

Provided herein are systems for monitoring mental health and/or detecting and/or predicting a depressive relapse using one or more models with one or more neural networks (e.g., a regression model, classification model, a recurrent deep neural network (RDNN), etc.). To monitor mental health and/or determine and/or predict a depressive relapse, certain information (e.g., data, features, indicators) may be determined to be significant with respect to detecting and/or predicting a relapse or otherwise monitoring mental health. Baseline values of such information may be determined for an individual which may represent typical and/or healthy values for the individual (e.g., when the individual is not experiencing a depressive episode).

Once baseline values are determined, deviations from the baseline may be routinely or periodically identified. The deviations may be processed by one or more models trained to determine a score indicative of depressive episode and/or a label indicative of a depressive level to detect and/or predict a depressive relapse. For example, the model may be a classification model, a regression model, and/or a recurrent deep neural network (RDNN) trained to determine a depression score indicative of a degree, severity, or level of depression.

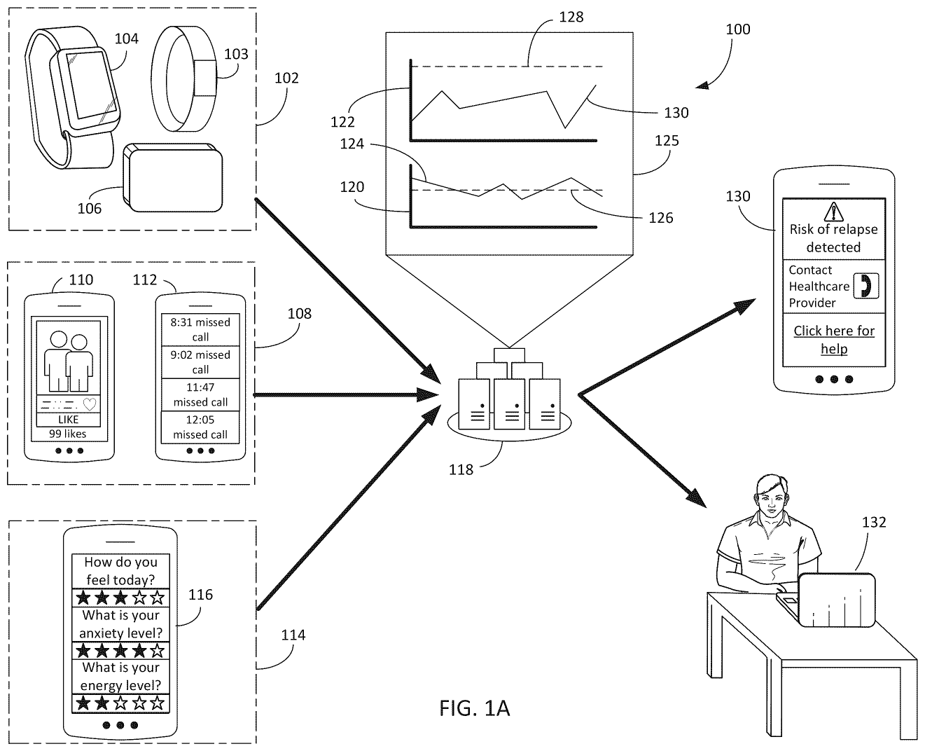

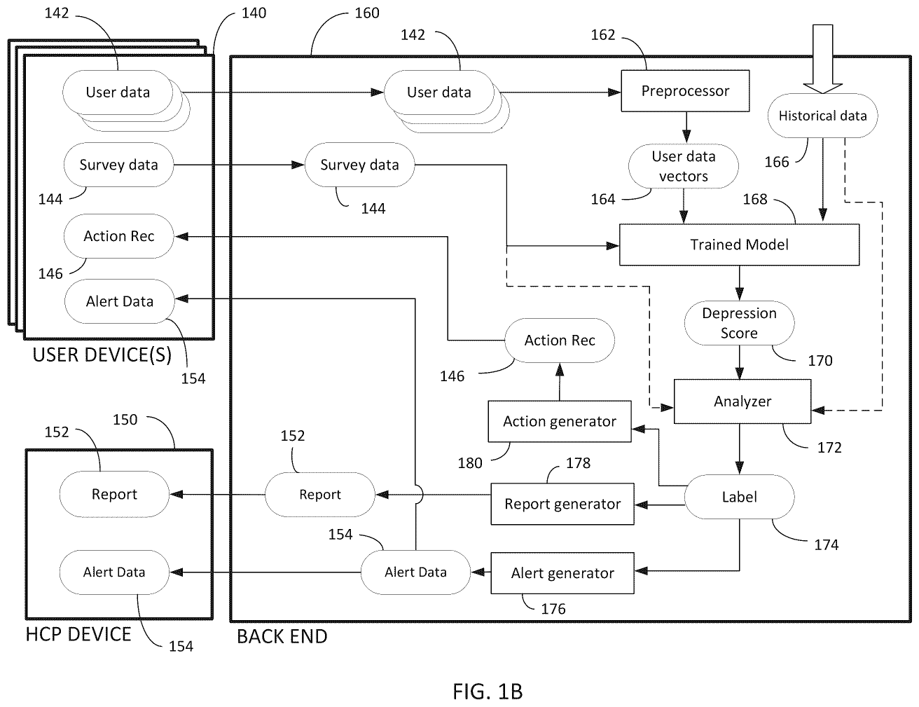

Referring now to FIG. 1A, a system for monitoring mental health and/or detecting and/or predicting a depressive relapse is illustrated and may include physiological devices, behavioral devices, survey devices, servers, user devices, and/or a healthcare devices. As shown in FIG. 1A, physiological devices, user devices, and/or survey devices may send physiological data, activity data, exercise data, cardiovascular data, sleep data, mood data, behavioral data, survey data, and/or other patient information to a server, which may be a remote server, to analyze the data, monitor the metal health of the patient, and/or determine and/or predict a risk of a depressive relapse. The risk of the depressive relapse may be sent to a healthcare provider device and/or a patient device.

Physiological devices 102 may include wearable, implantable, and/or other user devices. For example, physiological devices 102 may include smart watch 104, wearable device 103, medical device 106, and/or any other user device, electronic device, smart sensor, and/or other device for generating physiological information corresponding to the user. Physiological devices 102 may generate and/or determine physiological information and/or data such as heart rate information (beats per minute, heart rate variability, electrocardiogram (ECG) data etc.), temperature information, blood oxygenation information, respiratory rate information, sleep metrics (e.g., duration, time to fall asleep, time asleep, time awake, time after wakeup, time-in-bed, micro-awakenings, time deep sleep, time in deep wake, time in rapid eye movement (REM), etc.), physical activity (e.g., steps, sedentary time, active time, etc.), average beats per minute, breathing rate, sleep breathing rate, sleep temperature, time to fall asleep, time in bed, seconds in deep sleep, seconds in deep wake, sedentary time, unanswered text messages, average size of messages (e.g., number of words), unanswered calls, average length of calls, tone of voice, speech speed (e.g., words per minute), frequency of speech, verbal content, prosodic variability, pauses and hesitation, emotional reactivity, and/or the like. Medical device 106 may be any ambulatory or implantable medical device (e.g., insulin pump, continuous glucose monitor, pacemaker, etc.).

Behavioral devices 108 may include user devices 110 and/or 112 which may be smart phones, tablets, laptop devices, and/or any other computing devices (e.g., having memory and a processor) for viewing social media, making and receiving phone calls, sending and receiving emails and/or texts, accessing the internet via a web browser, and the like. Behavioral devices 108 may generate and/or determine behavioral information and/or data such as total screen time, total time spent on social media, number of unanswered messages, average size in message in number of words, number of unanswered calls, emails, and/or texts, average length of calls, and the like. Alternatively, or additionally, physiological device 102 and/or behavioral device 108 may include GPS or other positioning information, which may provide the location and/or whereabouts of the user, which may be compared to known locations such a home or work location.

Survey device 114 may include user device 116 which may be a smart phone, tablet, laptop device, and/or any other computing device (e.g., having memory and a processor). User device 116 may be the same as or different than user device 110 and/or 112. Survey device 114 may generate survey information and/or data about a user based on questions or prompts presented to the user via user device 116. For example, questions may include “how do you feel today,” what is your level of anxiety today,” “what is your energy level,” “what is your appetite,” what is your level of concentration,” and/or “what is your level of self-confidence.”

Physiological devices 102, behavioral devices 108, and/or survey device 114 may communicate, either directly or indirectly, with server 118 via any suitable wireless technology. Server 118 may be one or more, computing device (e.g., having memory and a processor), server, and/or data store. Server may further communicate with user device 130 which may be the same as user device 110, user device 112 and/or user device 116. Alternatively, or additionally server 118 may communicate with healthcare provider (HCP) device 132, which may be any smartphone, laptop, tablet, or other computing device used by a healthcare provider. Server 118 may communicate with user device 130 and/or healthcare provider device 132 via any suitable wireless technology.

As shown in FIG. 1A, physiological devices 102 may send physiological data to server 118, behavioral devices 108 may send behavioral data to server 118, and/or survey device 114 may send survey data to server 118. Server 118 determine baseline metrics for the data received by periphery devices 102, behavioral devices 108, and/or survey device 114. For example, average, minimum, maximum, standard deviation, and/or other metrics may be determined for such data over a given period of time (e.g., hours, days, weeks, months, etc.).

Server 118 may continuously, routinely, and/or periodically, receive physiological, behavioral, and/or survey data and may compare such data to the baseline values for real-time or near real-time tracking of mental health. For example, plot 122 may correspond to a heart rate of the individual over time and with heart rate values 130 compared to baseline 128 which may be an average heart rate value. Plot 120 may be sleep duration over time with sleep values 124 compared to baseline 126 which may be an average sleep value.

Once deviations from baseline are determined for physiological, behavioral, and/or survey data, server 118 may input this information into machine learning models trained to process the deviations and determine a depression score and/or label indicative of whether the individual is currently or is likely to in the near future experience a depressive episode. For example, a regression model may generate a depression score and/or a classification model may generate a depression label which may indicate a degree of depression.

The depression score and/or label may be compared against a threshold value and/or level to determine a risk of depressive event. If a certain threshold value or level is achieved or surpassed, indicating the presence of likelihood of a depressive event, server 118 may cause user device 130 to present the identified risk of a relapse of a depressive event and/or may send an alert indicating the risk of a relapse of a depressive event to healthcare device 132. User device may 130 optionally present a link or button to immediately contact a healthcare provider or emergency services, and/or other emergency contact information.

Referring now to FIG. 1B, a schematic view of the data flow between user devices, a healthcare provider (HCP) device, and back end of a mental health monitoring system is depicted. User devices 140 may include one or more of physiological devices 102, behavioral devices 108, and/or survey device 114 described above with respect to FIG. 1A. For example, user devices 140 may be any electrical and/or computer device that may generate information about the user, such as for example, heart rate data, ECG data, sleep data, temperature data, breathing data, cardiovascular data, activity data, step data, screen time data, media data, call data, text message data, email data, location data, and/or any other relevant information about or relating to the user.

HCP device 150 may be one or more device (e.g., electronic and/or computer device) which may be, for example, HCP device 132 described above with respect to FIG. 1A. Back end 160 may be any computing device with one or more processors capable of performing operations described herein (e.g., server 118). In the example illustrated in FIG. 1B, back end 160 may be one or more server, desktop or laptop computer, or the like and/or may be located in a different location than user device 140 and/or HCP device 132. Back end 160 may run one or more local applications and/or may include and/or communication with one or more datastore. User devices 140, HCP device 150 and/or back end 160 communicate via any well-known wired or wireless technology (e.g., Wi-Fi, cellular network, Bluetooth, Bluetooth Low Energy (BLE), near field communication protocol, etc.)

As shown in FIG. 1B, user devices 140 may generate user data 142 (e.g., heart rate data, ECG data, sleep data, temperature data, breathing data, cardiovascular data, activity data, step data, screen time data, media data, call data, text message data, email data, location data, and/or any other relevant information about or relating to the user) and/or survey data 144. Survey data 144 may include information or data generated in response to a survey or other prompts and/or feedback or other user input information. The survey data may correspond to and/or may be indicative of a depressive state of the user (e.g., a high, medium, or low depressive state). User device may send user data 142 and survey data 144 to back end 160 (e.g. via the Internet).

Back end 160 may receive user data 142 and process user data 142 using preprocessor 162. Preprocessor 162 may transform user data 142, which may be raw data generated by user devices 140 or otherwise may be representations of such data generated by user device 140, into user data vectors 164. User data vectors 164 may be vector representations of user data 142. In one example, preprocessor may be and/or implement an embedding algorithm (e.g., embedding neural network).

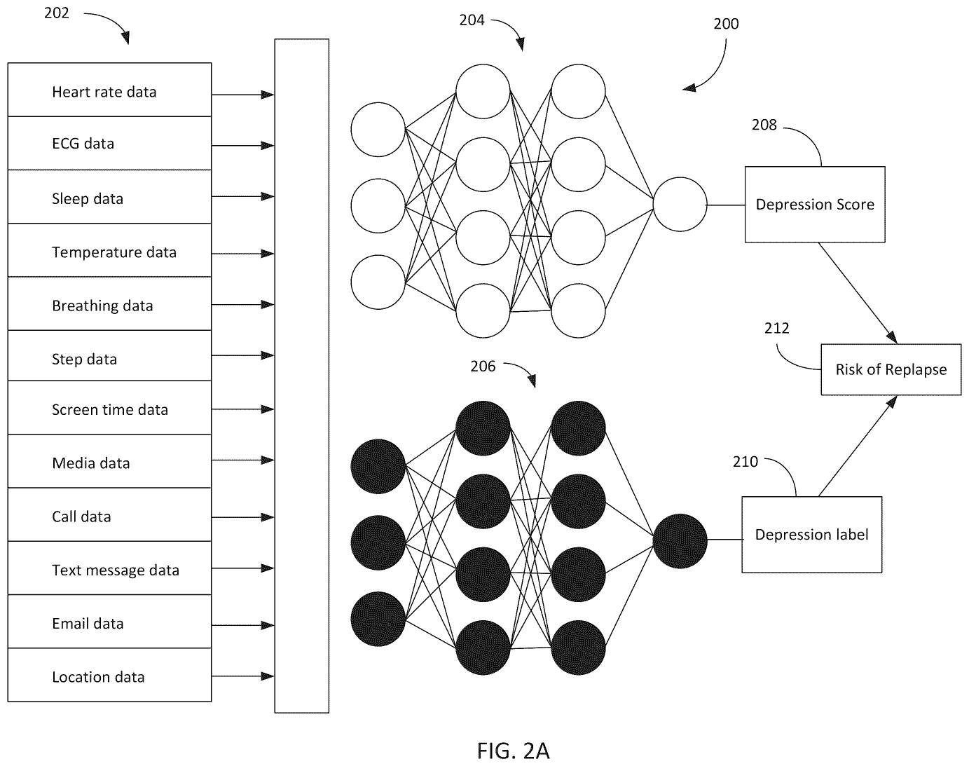

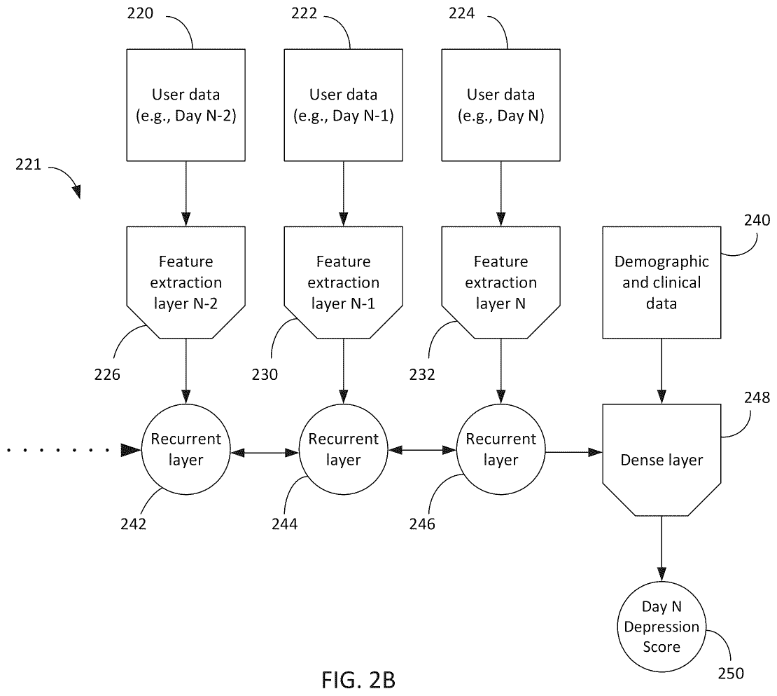

User data vectors 164 and/or user device 142 may be input into trained model 168 for processing. Additionally, survey data 144 and/or historical data 166 may optionally be input into trained model 168. Historical data 166 may be demographic data, clinical data, medical history data, or any other previously generated data relating to the user. Trained model 168 may be one or more neural networks. For example, trained model 168 may be regression model 204 and/or classification model 206 trained to generate a score indicative of a degree of depression of the user (e.g., severity of depression), a label (e.g., corresponding to a depression state or level), a risk of depression relapse, or any other information relating to the mental health of the user, as explained in more detail below with respect to FIG. 2A. Alternatively, or additionally, trained model 168 may be a recurrent deep neural network (RDNN), explained in more detail below with respect to FIG. 2B.

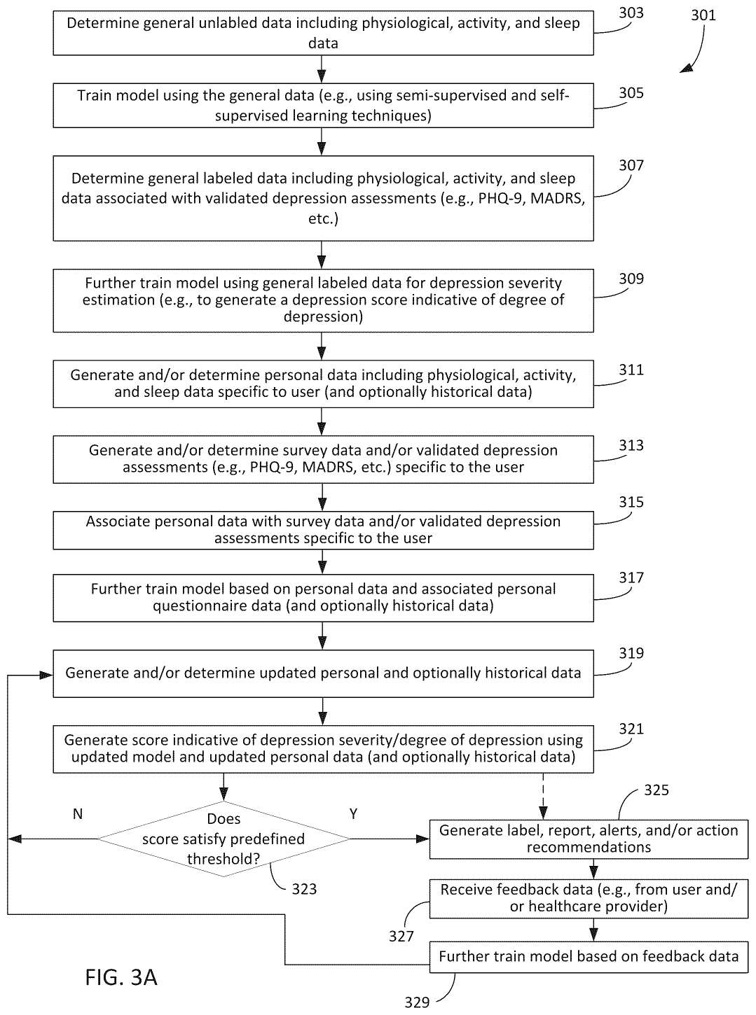

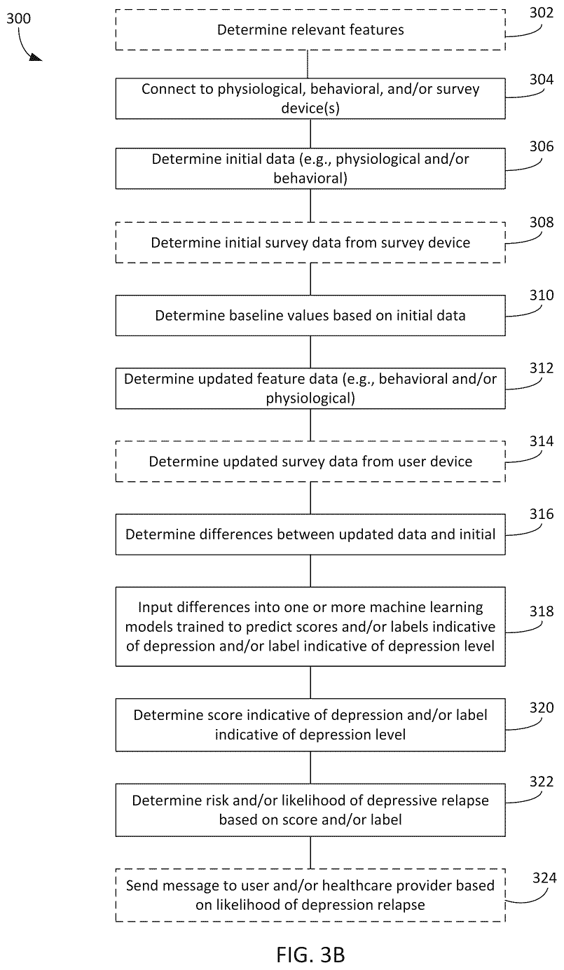

Trained model 168 may be trained using the process set forth below with respect to FIG. 3A. Survey data 144 generated by user device 140 may be shared with back end 160 (e.g., via the Internet) and may be used to train trained model 168. For example, survey data may include or represent a patient's assessment of the mental health at a given time and such assessment may be compared to collected user data and correlated to such user data at the given time. Survey data or other mental health assessment information may be additionally, or alternatively, be generated by HCP device 150 and shared with back end 160.

Trained model 168 may output depression score 170. Additionally, or alternatively, trained model 168 may output other information, such as a label or other mental health information. Depression score 170 may be indicative of severity, degree, and/or level of depression. In one example, depression score may be a number between 0 and 27 with 27 being the highest level of depression.

Depression score 170 may be input into analyzer 172, which may process depression score 170 and generate label 174, which may be a relapse label, for example. Analyzer 172 may be one or more algorithms that may compare depression score 170 and/or other information output from trained model 168 to one or more predefined threshold values or other trends or the like. For example, analyzer 172 may compare depression score 170 to a predefined threshold value or a range or spectrum of threshold values. In one example, a predefined spectrum of threshold values may be stored on back end 160 (e.g., a threshold value for low risk of depression relapse, a threshold value for medium risk of depression relapse, and a depression value for high risk of depression relapse).

Label 174 may be indicative of whether the risk of depression relapse is low, medium, or high. Alternatively, or additionally, label 174 may indicate whether the user is currently depressed and/or the level or severity of the depression (e.g., low depressed state, medium depressed state, or high depressed state).

Alternatively, or additionally, label 147 may be indicative of stagnation, aggravation, and/or recurrence. For example, if the patient is determined to be in an acute phase a depression, then one of two labels may be generated-aggravation or stagnation. If the depression score increases by more than a certain amount of points starting from the first week of care, or other suitable time frame, an aggravation label may be generated. Alternatively, if the depression score is equal two or exceeds a predetermined threshold starting from the fourth week of care, or other suitable time frame, a stagnation label may be generated. In another example, if the patient is in the remission phase (e.g., the depression score is below a threshold starting from the first week of remission), one alert that may be generated is the relapse alert. The relapse alert may be generated if the depression score exceeds a certain threshold value during the remission phase. In yet another example, if the patient is in a recovery phase (e.g., depression score is below a threshold value starting from the first week of recovery), a recurrence label may be generated if a new depressive episode is detected (e.g., depression score is a above a threshold valve indicative of a depressive episode) during the recovery phase. The foregoing list of labels is non-limiting and it will be understood by one skilled in the art that other suitable depression labels may be generated.

Label 174 may then be processed by alert generator 176, report generator 178, and/or action generator 180. Alert generator 176 may be one or more algorithms that may receive label 174 or optionally depression score 170 and determine whether or not to generate alert data 154, which may include an alert indicating label 174 or optionally depression score and/or information relating thereto. Alert data 154 may be sent to one or more of user devices 140 or HCP device 150. In one example, any label 174, or optionally depression score 170, may cause alert generator 176 to generate alert data 154, which may include an aggravation alert, a stagnation alert, relapse alert, or recurrence alert, for example. Alternatively, only certain labels or optionally depression scores (e.g., high risk of relapse labels, severe depression label, high depression score), may cause alert generator 176 to generate alert data 154. User device 140 and/or HCP device 154 may be caused to display the alert data.

Report generator 178 may be one or more algorithms that may receive label 174 or optionally depression score 170 and may generate report 152 with such label 174 and/or depression score 170. Back end 160 may send report 152 to HCP device 150 and/or one or more of user devices 140. Report 152 may include depression scores, labels, user data, and calculations and/or charts, plots, and/or representations of such data over a certain period of time.

Action generator 180 may be one or more algorithms that may receive label 174 or optionally depression score 170 and may generate action recommendation 146, which may be sent to one or more user device 140 for display on such user device (e.g., backend 160 may cause user device 140 to display such action recommendation). Action recommendation 146 may be one or several treatment recommendations or recommendations to perform certain actions to alleviate, treat, address, or otherwise improve the mental health of the user and/or may be based on alert 174. For example, action recommendation 146 may be a suggestion to exercise, to perform a certain exercise, to get more sleep, to schedule an appointment with a healthcare provider (e.g., psychiatrist), make changes to the user's diet, go outside, stop using social media, meet with friends and/or family, or perform other actions or activities to improve the user's mental health. Some or all of the operations of the data flow between user devices, a healthcare provider (HCP) device, and back end of a mental health monitoring system may be optional and may be performed in a different order.

Referring now to FIG. 2A, a schematic view of depressive relapse detection and/or prediction system 200 having one or more neural networks is illustrated. For example, inputs 202 may be input into one or more models trained to determine a score and/or label indicative of presence of a depressive state or risk of depressive state. Inputs 202 may be the raw data itself and/or the difference between physiological, behavioral, and/or survey values indicative of a current state of the individual and baseline physiological, behavior, and/or survey values and/or any other stored values (e.g., values from an earlier time point). For example, inputs 202 may be deviations from bassline values of beats per minimum. In one example, inputs may include, without limitation, heart rate data, ECG data, sleep data (e.g., amount of (rapid eye movement (REM) sleep), temperature data, breathing data, step data, screen time data, media data (e.g., information about the type of media content consumed and/or the amount of time consumed), call data, text message data, email data, location data (e.g., GPS location), heart rate variability, hours of sleep, breathing rate, number of steps or other exercise and/or cardiovascular information, total screen time, social media screen time, number of missed calls, number of unanswered text messages, number of unanswered emails, and/or length of calls, or the like. It is understood that inputs 202 may alternatively or additionally be any other features, data, information. Alternatively, inputs 202 may be raw data received from periphery devices, behavioral devices, and/or survey devices or any representation and/or portion thereof.

Inputs 202 may be input into regression model 204 and/or classification model 206. Regression model 204 may be one or more regression neural networks for detecting and/or predicting severity of depression by outputting a depression score (e.g., a patient health questionnaire score (PHQ)-9 score). Classification model 206 may be one or more classification neural networks for detecting and/or predicting severity of depression, which outputs a label indicating a category of depression (e.g., absent or minimal depressive disorder, mild or subthreshold depressive disorder, marked depressive disorder, severe depressive disorder, etc.).

While both regression model 204 and classification model 206 are illustrated in the depressive relapse detection and/or prediction system, it is understood that only one model may be used in the system. Additionally, or alternatively, other models may be used such as linear models (e.g., logistic regression models), single tree models (decision trees), ensemble models (e.g., gradient boosting models), and/or deep learning models (e.g., multi-layer perceptron (MLP) models), and/or any other suitable models.

Classification model 206 may output depression score 208 which may be a score ranging from 0 to 27, for example, which may be indicative of severity of depression (e.g., degree or level of depression). For example, 27 may be the highest severity of depression on a scale of 0 to 27. Regression model may output a label which may indicate the severity of depression. For example, a set number (e.g., 5) of distinct labels may be used and may each corresponding to a range of scores (e.g., between 0 to 27). It is understood that any other scoring scheme and/or label scheme may be used (e.g., low depression, medium depression, severe depression).

Depression score 208 and depression label 210 may be used to determine risk of depressive relapse 212. For example, the risk of depressive relapse may be determined by comparing the score from depression score 208 and/or depression label 210 to a threshold value and/or threshold label or severity level and if the threshold score and/or level is satisfied and/or exceeded, a high risk of relapse may be determined and/or the presence of relapse may be determined. Different threshold values and/or levels may indicate a low or medium risk of relapse. Based on the risk of depressive relapse, the depressive relapse detection and/or prediction system 200 may send a message to a user device and/or healthcare provider device providing risk of relapse score 212, depression score 208, and/or depression label 210.

Referring now to FIG. 2B, an exemplary neural network including a schematic view of a mental health monitoring model using one or more neural networks is illustrated. For example, FIG. 2B illustrates user data (e.g., preprocessed user data transformed into a single vector) input into neural network 201, which may be a recurrent deep neural network (RDNN). User data 220 may correct day N−2, user data N−1 may correspond to day N−1 and user data 224 may correspond day N. It is understand that discrete user data may correspond to different time periods (e.g., one hour, one week, one month, etc.). User data 220, 222 and/or 224 may correspond to one or more data modalities.

Neural network 201 may include multiple feature extraction layers (e.g., feature extraction layers 226, 230, and 232). Feature extraction layers 226, 230, and 232 may be applied independently to user data 220, 222, and 224, respectively. For example, dense and/or convolution layers may be applied independently to each daily vector to product a daily representation of the data. In one example, an independent feature extraction layer may be applied to each set of user data (e.g., to each single daily vector representing the data collected for a given day). Each of feature extraction layer 226, 230, and 232 may generate and/or product an output that is representation of the user data input into the respective feature extraction layer.

After the feature extraction layer, the output of each feature extraction layer (e.g., feature extraction layers 226, 230, and/or 232) may be input into a respective recurrent layer (recurrent layers 242, 244, and/or 246). Recurrent layers 242, 244, and 246 may be long short-term memory (LSTM), transformers, and/or any other suitable recurrent layers. Recurrent layers 242, 244, and 246 process the output of the feature extraction layers to capture temporal dependencies and/or relationships across user data (e.g., user data 220, 222, and 224). For example, recurrent layers may capture temporal dependencies across the sequence of daily vectors representing the user data.

The output of the recurrent layers (e.g., recurrent layers 242, 244, and 246) may be concatenated with historical data, which may include without limitation user demographic and clinical data, such as age, sex, previous psychiatric diagnostics, medical history data, family history data, and/or any other relevant historical and/or other relevant user data and may together be processed by dense layer 248. Dense layer 248 may be a fully connected layer in which each neuron or node in the layer may be connected to every neuron or node in the previous layer. Dense layer 248 may generate depression score 250 which may be a degree, level and/or severity of depression. For example, depression score may correspond to the most recent day or other time period corresponding to the user data (e.g., the most recent user data corresponds to day N and the depression score also corresponds to day N).

Referring now to FIG. 3A, a process flow is depicted for training one or more models trained to output scores and/or labels indicative of a severity of depression. Some or all of the blocks of the process flow in this disclosure may be performed in a distributed manner across any number of devices (e.g., a server such as server 118 of FIG. 1, computing devices, user devices, periphery devices, healthcare provider devices, or the like). Some or all of the operations of the process flow may be optional and may be performed in a different order.

To initiate process flow 301, at block 303, computer-executable instructions stored on a memory of a device, such as a server, may be executed to determine general (e.g., not specific to a single user) unlabeled data including physiological data, activity data, and/or sleep data relevant to the mental health one or more individuals different than the user. The unlabeled data may not be associated with a mental state or degree of depression. The data may be any data mentioned above with respect to FIG. 2A and/or otherwise any data relevant to mental health. At block 305, computer-executable instructions stored on a memory of a device, such as a server, may be executed to train a model (e.g., a machine learning algorithm) using the general data. For example, the model may include one or more neural networks and semi-supervised and/or self-supervised learning techniques may be used to train the model.

At block 307, computer-executable instructions stored on a memory of a device, such as a server, may be executed to determine general labeled data including physiological, activity, and sleep data corresponding to one or more individuals different than the user and associated with validated depression assessments (e.g., Patient Health Questionnaire (PHQ)-9, Montgomery-Åsberg Depression Rating Scale (MADRS), etc.). For example, the data may be associated with depression scores, labels, and/or levels at a given time corresponding to the data or otherwise may be associated with the data. The data may be any data mentioned above with respect to FIG. 2A and/or otherwise any data relevant to mental health. The assessments may be generated by the individual and/or the healthcare professional. At block 309, computer-executable instructions stored on a memory of a device, such as a server, may be executed to further train the model using the general labeled data to finetune the model to generate a depression severity estimation. For example, the output may be a depression score indicative of a degree of depression for the user at a given point in time.

At block 311, computer-executable instructions stored on a memory of a device, such as a server, may be executed to generate and/or determine personal data including physiological, activity and/or sleep data specific to the user, as well as optionally historical data (e.g., historical data 240 of FIG. 2B). The data may be any data mentioned above with respect to FIG. 2A and/or otherwise any data relevant to mental health. At block 313, computer-executable instructions stored on a memory of a device, such as a server, may be executed to generate and/or determine survey data and/or other validated depression assessment data (e.g., PHQ-9, MADRS, etc.) specific to the user.

At block 315, computer-executable instructions stored on a memory of a device, such as a server, may be executed to associate personal data with survey data and/or validated depression assessments specific to the user. For example, the survey data and/or validated depression assessment data specific to the user may be associated with a time point or period of time and each of the personal data (e.g., personal user data) may be similarly associated with a time point or period of time to associate the survey data and/or validated depression assessment data specific to the user with the personal data. Alternatively, other suitable techniques may be used to associate the survey data and/or validated depression assessment data specific to the user with the personal data.

At block 317, computer-executable instructions stored on a memory of a device, such as a server, may be executed to further train the model based on the personal data generated at block 311 and the survey data and/or survey data or other validated depression assessments generated at block 313. At block 319, computer-executable instructions stored on a memory of a device, such as a server, may be executed to determine updated personal data and optionally historical data. For example, the personal data may be any data mentioned above with respect to FIG. 2A and/or otherwise any data relevant to mental health and may be associated with a time point or time period. The time point of the updated personal data at block 319 may be later in time that a time point associated with the personal data at block 311.

At block 321, computer-executable instructions stored on a memory of a device, such as a server, may be executed to generate a score indicative of depression severity (e.g., degree or level of depression) using updated model and updated personal data (and optionally historical data). The score may correspond to the same time point as the time point associated with the updated personal data at block 319. For example, the score may be the same as depression score 250 of FIG. 2B. At decision 323, computer-executable instructions stored on a memory of a device, such as a server, may be executed to determine whether the score generated at block 321 satisfies a predetermined threshold value. For example, at decision 323, the score may be compared to a single threshold value or a spectrum of threshold values to determine if the score is above a certain threshold value or within a range of threshold values (e.g., above a threshold value indicative of a high degree of depression).

If the score does not satisfy the predetermined threshold or thresholds, the mental health monitoring system may be programmed to reinitiate block 319 to generate and/or determine updated personal data. If instead the score does satisfy the predetermined threshold or thresholds, block 325 may be initiated. Alternatively, block 325 may optionally be initiated after block 321, whether or not the score satisfies the threshold or thresholds. At block 325, computer-executable instructions stored on a memory of a device, such as a server, may be executed to generate a label, report, alert, and/or recommend an action. For example, at block 325, a lapse such as relapse label 174 of FIG. 1B may be generated or determined, alert data such as alert data 154 of FIG. 1B may be generated or determined, a report such as report 152 of FIG. 1B, and/or an action recommendation such as action recommendation 146 of FIG. 1B may be generated or reported. Any of the label, report, alert, and/or action recommendation may be sent to one or more user device and/or HCP device for display on such device or devices.

At block 327, computer-executable instructions stored on a memory of a device, such as a server, may be executed to receive feedback data (e.g., from user and/or healthcare provider). For example, upon receiving any of the label, report, alert, and/or action recommendation, a user and/or healthcare provider may send feedback or other input to the mental health monitoring system (e.g., feedback data). Such feedback may indicate that any of the label, report, alert, and/or action recommendation were wrong, incorrect, or inaccurate for example. At block 329, computer-executable instructions stored on a memory of a device, such as a server, may be executed to further train the model based on the feedback received at block 327. After block 329, block 319 may be reinitiated.

Referring now to FIG. 3B, a process flow is depicted for determining a depressive relapse illustrated. Some or all of the blocks of the process flow in this disclosure may be performed in a distributed manner across any number of devices (e.g., a server such as server 118 of FIG. 1B, computing devices, user devices, periphery devices, healthcare provider devices, or the like). Some or all of the operations of the process flow may be optional and may be performed in a different order.

To initiate process flow 300, at optional block 302, computer-executable instructions stored on a memory of a device, such as a server, may be executed to determine relevant information, data, and/or features. Relevant data, information, and/or features may be physiological, behavioral, survey, and/or any other data that may change between normal and depressive states of an individual and may be significant with respect to detecting a depressive relapse and/or event. At block 304, computer-executable instructions stored on a memory of a device, such as a server, may be executed to connect to physiological, behavior, and/or survey devices, either directly or indirectly via one or more other devices.

At block 306, computer-executable instructions stored on a memory of a device, such as a server, may be executed to determine initial data (e.g., physiological and/or behavioral data) from physiological and/or behavioral devices. At optional block 308, computer-executable instructions stored on a memory of a device, such as a server, may be executed to determine initial survey data from a survey device. At block 310, computer-executable instructions stored on a memory of a device, such as a server, may be executed to determine baseline values based on the initial data (e.g., initial physiological, behavioral, and/or survey data).

At block 312, computer-executable instructions stored on a memory of a device, such as a server, may be executed to determine updated data (e.g., updated physiological and/or behavioral data from physiological and/or behavioral devices). At optional block 314, computer-executable instructions stored on a memory of a device, such as a server, may be executed to determine updated survey data (e.g., updated survey data from survey devices).

At block 316, computer-executable instructions stored on a memory of a device, such as a server, may be executed to determine differences and/or deviations between updated data (e.g., updated physiological, behavioral, and/or survey data) and initial data (e.g., initial physiological, behavioral, and/or survey data). At block 318, computer-executable instructions stored on a memory of a device, such as a server, may be executed to input the differences into one or more machine learning models trained to predict scores and/or labels that are indicative of depression and/or a relapse or occurrence of a depressive event.

At block 320, computer-executable instructions stored on a memory of a device, such as a server, may be executed to determine scores and/or labels that are indicative of depression and/or a relapse or occurrence of a depressive event using the one or more machine learning models. At block 322, computer-executable instructions stored on a memory of a device, such as a server, may be executed to determine a risk and/or likelihood of a depressive relapse based on the score and/or label output from the one or more machine learning models. For example, the score and/or label may be compared against threshold scores and/or labels to determine severity level and/or risk of a depressive relapse and/or the occurrence of a depressive event. At block 324, computer-executable instructions stored on a memory of a device, such as a server, may be executed to send a message and/or information to a healthcare provider device and/or user device based on the risk and/or likelihood of a depressive relapse and/or based on the score and/or label output by the one or more machine learning models.

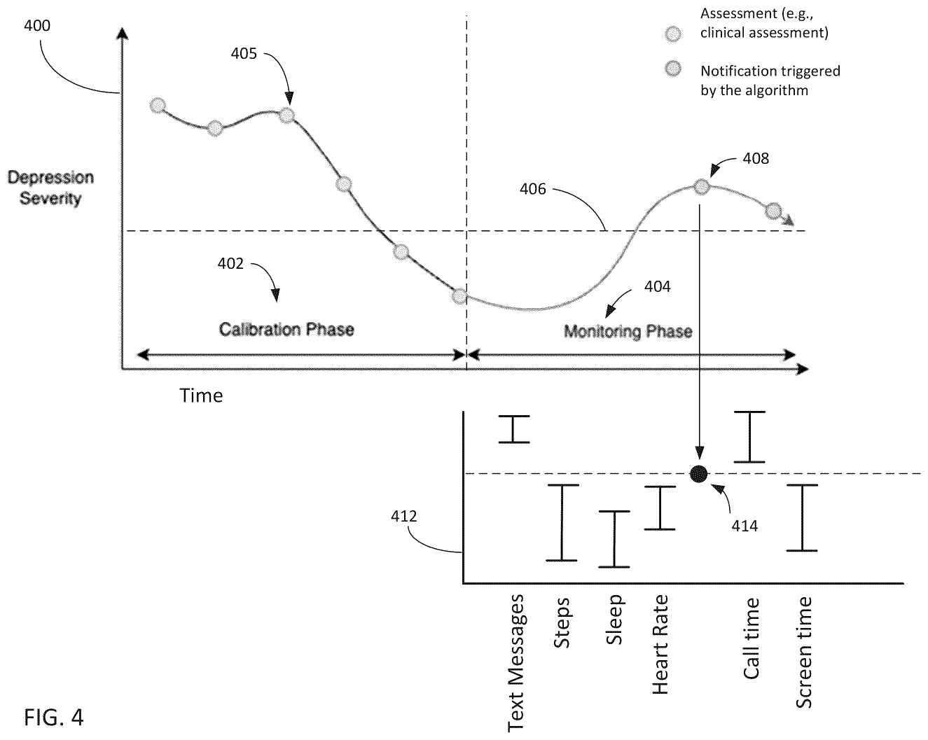

Referring now to FIG. 4, a plot of calibration and monitoring data as well as threshold values corresponding to healthy mental state for the patient is illustrated. As shown in FIG. 4, plot 4 illustrates depression scores plotted chronologically. Calibration phase 402 may be a time period for which the machine learning model is trained using user data of the patient and survey and/or assessment data corresponding the user during the same time period. For example, depression scores 405 may be based on clinical and/or patient assessments (e.g., surveys). In one example, the calibration phase may be 1 to 3 weeks. Monitoring phase 404 may occur once the calibration phase is complete. For example, depression score 408 may occur during monitoring phase 404, which may occur after calibration phase 402, and may be generated using the trained algorithm.

Threshold 406 may be a predetermined threshold value corresponding to a depression score corresponding to a high depression severity. As shown in FIG. 4, depression score 408 may be above threshold 406. Accordingly, depression score 408 may correspond to a time point at which the depression severity is deemed to be high and/or concerning and depression score 415 may correspond to a timepoint at which the depression severity is deemed to be less concerning and/or not considered high.

Depression score 408 may be determined to be above a certain severity threshold because the user data processed by the trained model to generate the depression score falls outside normal ranges deemed to correspond to normal depressive states. For example, user data 414 shown in plot 412 may correspond to depression score 408 and may be one or more of text message data, step data, heart rate data, call time data, and screen time data that may be processed by the trained model to generate depression score 408. As shown in plot 412, user data 414 may fall outside maximum and minimum values of such user data and thus may correspond to a depression severity level that is deemed to be outside a normal or healthy range.

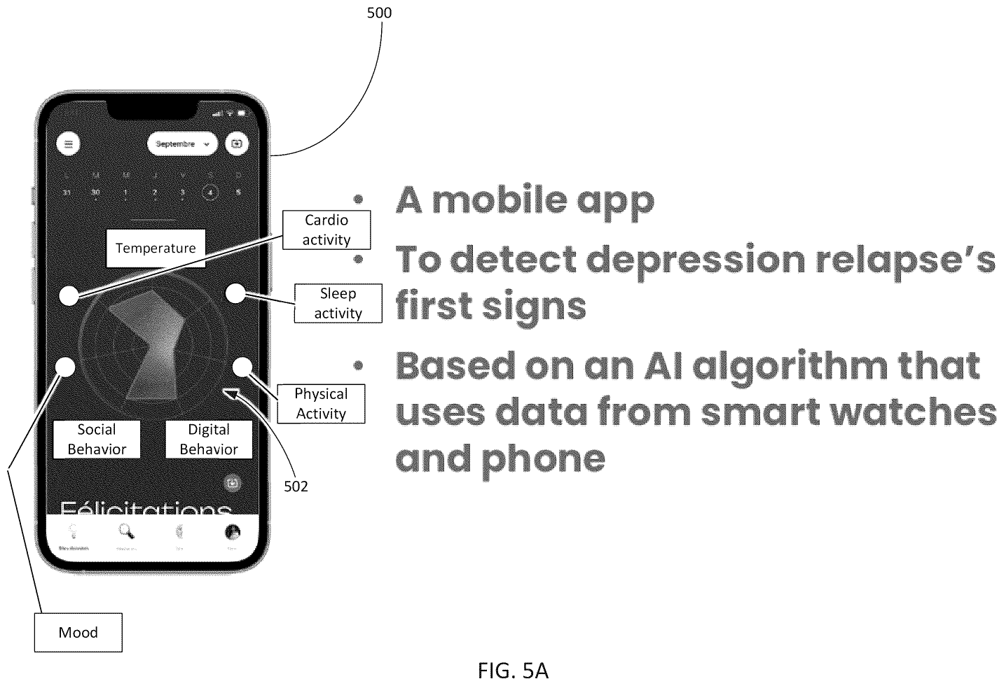

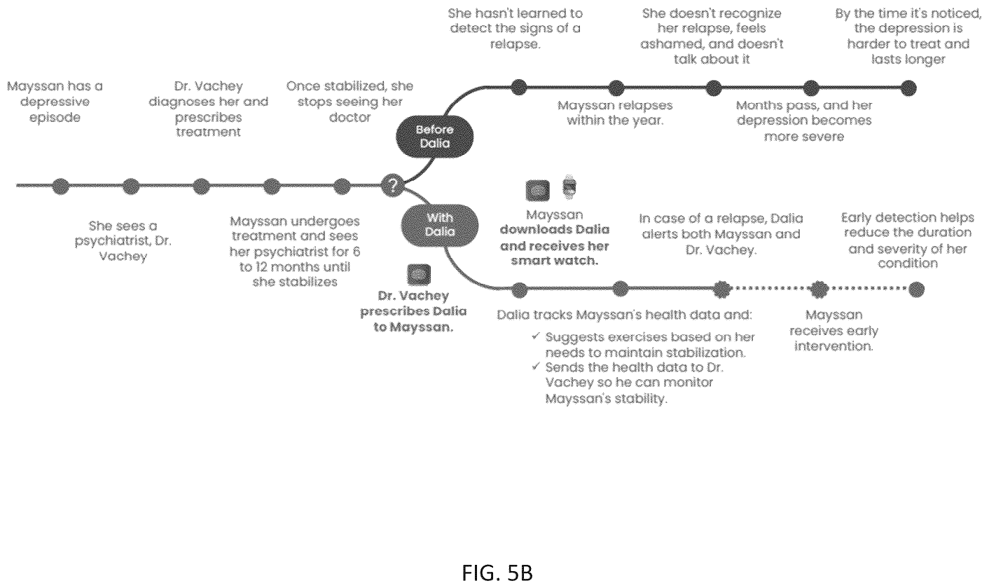

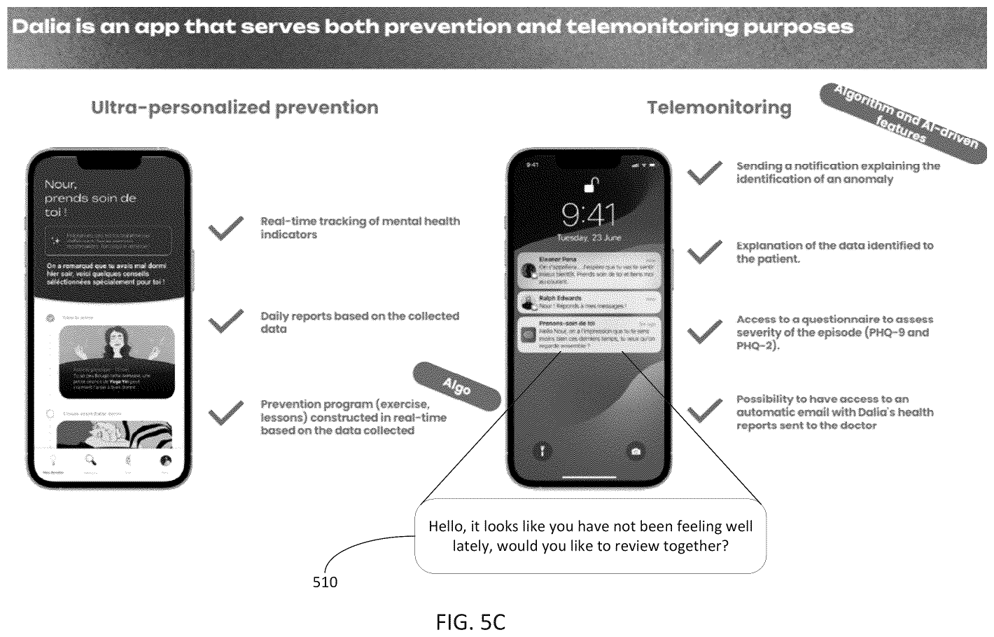

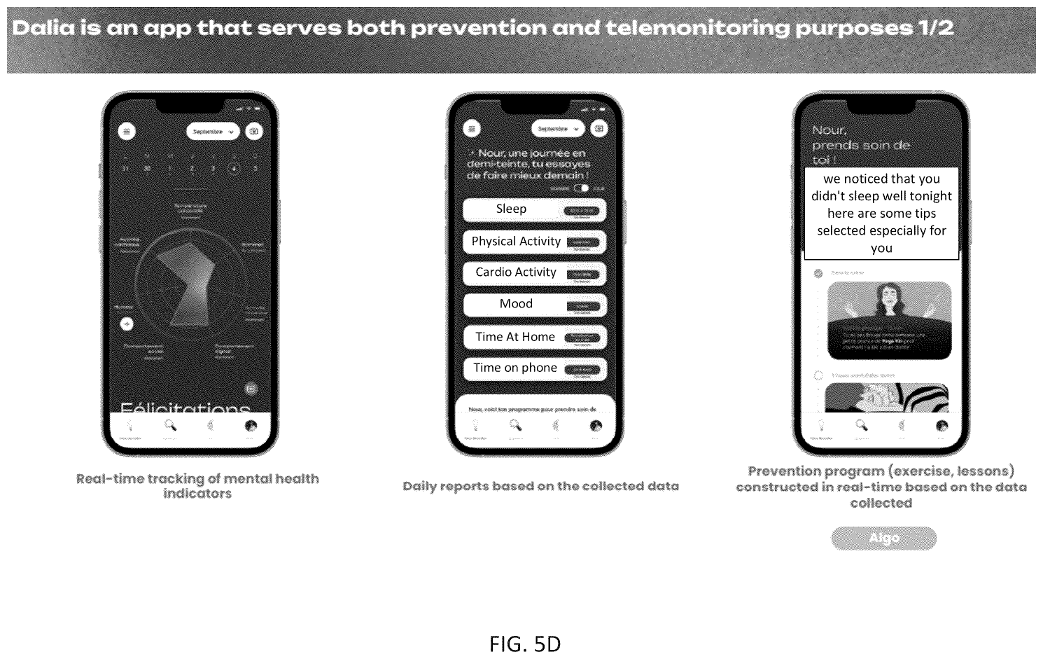

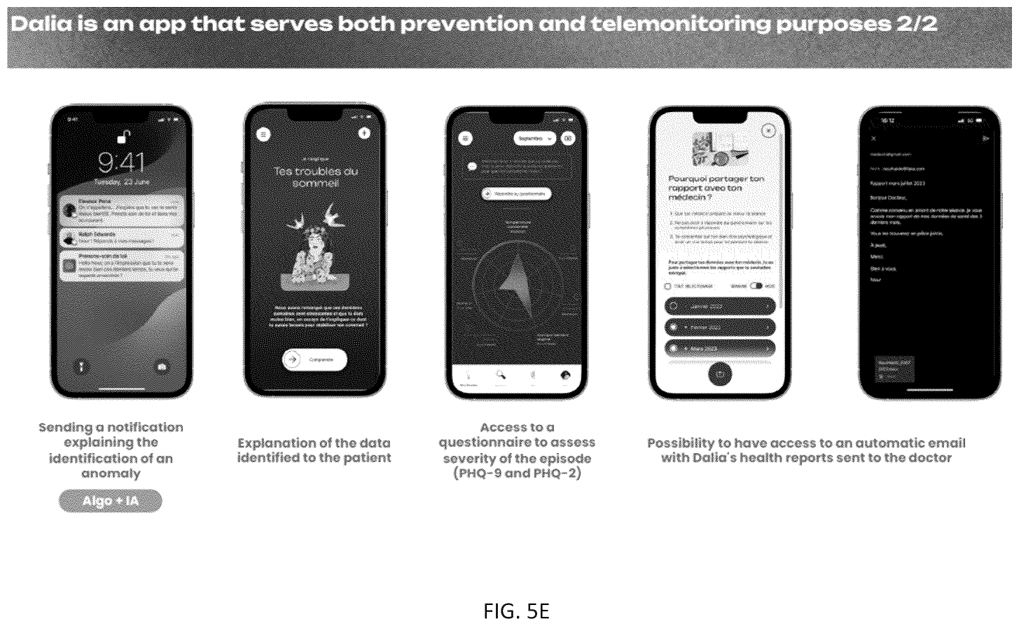

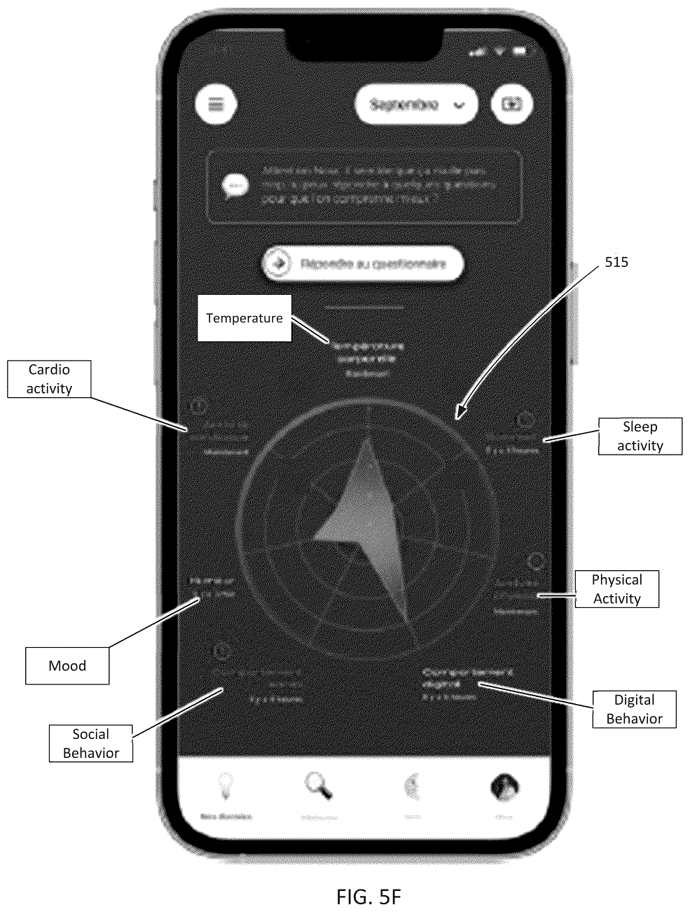

Referring now to FIGS. 5A-5F, exemplary graphic user interfaces and key points directed to the system for detecting and/or predicting a depressive relapse are provided. For example, in FIGS. 5A-5F exemplary graphic user interfaces including messages, information, and navigation of the depressive relapse detection and/or prediction system are illustrated as well as other information regarding the data flow, input information, output information, and data processing of the system for detecting and/or predicting a depressive relapse are illustrated. For example, FIG. 5A illustrates user interface 500 on a user device, which includes circular map 502 showing data relevant to a depressive state such as temperature, physical activity (e.g., amount of steps and/or movement to different locations), social behavior (e.g., time on social media), digital behavior (e.g., time on phone), cardio activity (e.g., exercise activity), and mood indicators (e.g., laughing in speech or text, smiling in photos, upbeat speech and/or text, etc.). Circular map 502 is described in more detail below with respect to FIG. 5F.

FIG. 5B illustrates the effects on the mental health of a user without using the mental health monitoring system compared to the mental health effects on a user using the mental health monitoring system. As shown in FIG. 5B, the user that does not use the mental health monitoring system does not recognize signs of depression relapse and several months may pass with their depression becoming more severe and harder to treat. The user that does use the mental health monitoring system receives alerts of the relapse or risk of relapse and receives early intervention that helps reduce the duration and/or severity of the condition.

Referring now to FIG. 5C, the mental health monitoring system may provide personalized prevention information as may telemonitoring functionality. For example, notifications explaining the identification of anomaly (e.g., risk of relapse, risk of depressive episode, current depression state, etc.) may be sent to the user and, optionally, an explanation of the data identified to the patient. For example, a message stating “hello, it looks like you have not been feeling well lately, would you like to review together,” may be sent to a user device of the patient, such as a mobile phone. Using the user device, a user may access email health reports that were sent to a healthcare provider.

Referring now to FIG. 5D, the mental health monitoring system may provide real-time tracking of mental health indicators, may provide daily reports based on the collected. For example, a graphic user interface may be generated with sleep data, physical activity data, cardiovascular data, mood data, time at home data, and/or time on the phone data. Prevention and/or treatment recommendations and/or programs may be presented on the user device. For example, exercise classes and/or lessons may be provided (e.g., yoga classes) based on the data provided. In one example, the system may detect a lack of sleep and suggest exercises known to improve quality of sleep.

Referring now to FIG. 5E, prevention and telemonitoring user interfaces of the mental health monitoring system displayed on a user device are illustrated. As shown in FIG. 5E, the mental health monitoring system may cause the user device to display a notification explaining results generated, explanation of data identified to the user, may provide access to a questionnaire (e.g., PHQ-9 and/or PHQ-2), and/or may provide messaging (e.g., text, email, etc.) with healthcare providers and/or other experts.

Referring now to FIG. 5F, a circular user data map is illustrated displayed on a user device. The circular map 520 may be a circular map having radial lines extending from the center to the perimeter of the map. Each radial line corresponding to a different data modality. Each data modality may be calibrated along the radial lines such that data plotted near the center of the circle represents a concerning amount of such user data (e.g., corresponding to a more severe depression state, level, or severity) and data plotted near the perimeter of the circle may correspond to an amount of user data that corresponds to a healthy or otherwise not concerning amount of such user data (e.g., corresponding to a less severe depression state, level, or severity). For example, plotted data near the center may be indicative of a depressive episode and data plotted data near the center of the circle may be indicative of a stable mental health state. Deviations from baseline or normal ranges of user data along the radial lines may be illustrated in the map using a color scheme (e.g., red/yellow corresponding to a significant deviation from baseline and/or normal range and green/blue corresponding to less significant deviation from baseline and/or normal range). In the example illustrated in FIG. 5F, the data modalities include cardio activity, mood data, social behavior, digital behavior, physical activity, sleep activity, and temperature, however it is understood that other data modalities and/or other types of user data may be alternatively or additionally included in the plot. As shown in FIG. 5, the text corresponding to data modalities with plotted data that is closest to the center may be shown in orange or red, if more severe. Text corresponding to data plotted closest to the perimeter of the circle may be shown in white or green.

In one example, the mental health monitoring system may be prescribed by a healthcare professional after stabilizing a patient after a depressive episode. The mental health monitoring system may improve mental health of users by improving stabilization of the user and/or detecting a potential relapse. Once a relapse is detected, healthcare providers (e.g., psychiatrists and/or general practitioners) may be consulted. Early patient care may reduce costs for healthcare providers and insurance companies and may permit optimization of the use of medical resources and better risk management.

The mental health monitoring system may provide users with a reduction of stress, easier access to care, and/or empowerment. The mental health monitoring system may provide healthcare providers with time optimization, early detection, and enhanced monitoring tools, and extension of the scope of care. The mental health monitoring system may also reduce healthcare (e.g., hospital) costs, optimize use of medical resources, and improve the quality of patient care.

With respect to health insurance, the mental health monitoring system may improve risk management, and reduce insurance claims (e.g., reduce length of work absences), for example.

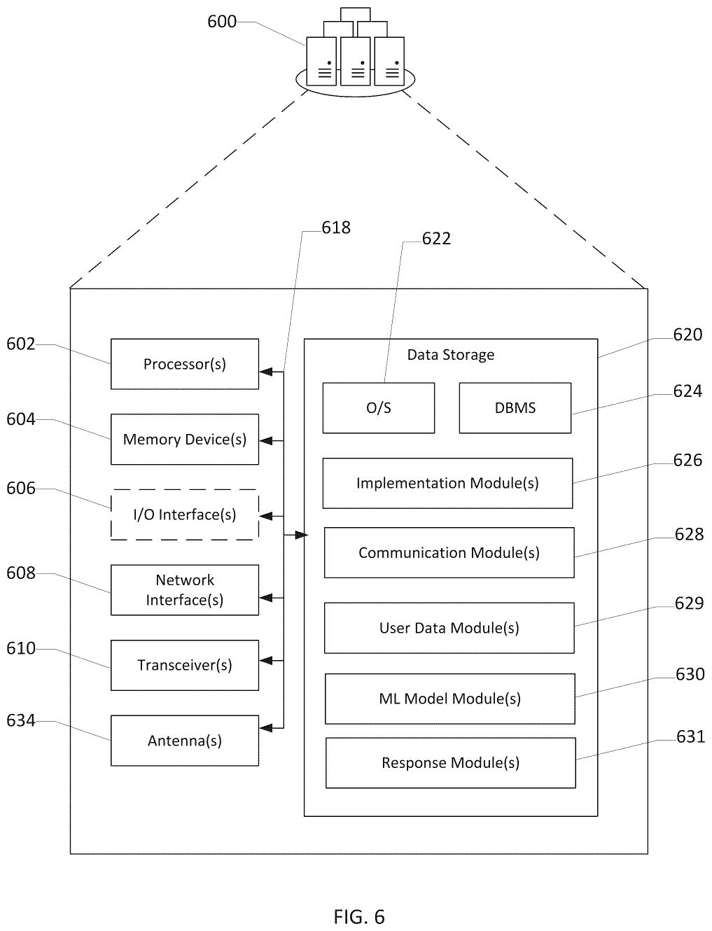

Referring now to FIG. 6, a schematic block diagram of server 600 is illustrated. Server 600 may be the same or similar to server 118 of FIG. 1A or otherwise one or more of the servers of FIGS. 1A-5F. It is understood that server 600 may alone or together with any of the user devices and/or healthcare provide devices perform one or more of the operations of server 600 described herein.

Server 600 may be designed to communicate with one or more servers, user devices, healthcare provider devices, data stores, other systems, or the like. Server 600 may be designed to communicate via one or more networks. Such network(s) may include, but are not limited to, any one or more different types of communications networks such as, for example, cable networks, public networks (e.g., the Internet), private networks (e.g., frame-relay networks), wireless networks, cellular networks, telephone networks (e.g., a public switched telephone network), or any other suitable private or public packet-switched or circuit-switched networks.

In an illustrative configuration, server 600 may include one or more processors 602, one or more memory devices 604 (also referred to herein as memory 604), one or more input/output (I/O) interface(s) 606, one or more network interface(s) 608, one or more transceiver(s) 610, one or more antenna(s) 634, and data storage 620. The server 600 may further include one or more bus(es) 618 that functionally couple various components of the server 600.

The bus(es) 618 may include at least one of a system bus, a memory bus, an address bus, or a message bus, and may permit exchange of information (e.g., data (including computer-executable code), signaling, etc.) between various components of the server 600. The bus(es) 618 may include, without limitation, a memory bus or a memory controller, a peripheral bus, an accelerated graphics port, and so forth. The bus(es) 618 may be associated with any suitable bus architecture including.

The memory 604 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In various implementations, the memory 604 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth.

The data storage 620 may include removable storage and/or non-removable storage including, but not limited to, magnetic storage, optical disk storage, and/or tape storage. The data storage 620 may provide non-volatile storage of computer-executable instructions and other data. The memory 604 and the data storage 620, removable and/or non-removable, are examples of computer-readable storage media (CRSM) as that term is used herein. The data storage 620 may store computer-executable code, instructions, or the like that may be loadable into the memory 604 and executable by the processor(s) 602 to cause the processor(s) 602 to perform or initiate various operations. The data storage 620 may additionally store data that may be copied to memory 604 for use by the processor(s) 602 during the execution of the computer-executable instructions. Moreover, output data generated as a result of execution of the computer-executable instructions by the processor(s) 602 may be stored initially in memory 604, and may ultimately be copied to data storage 620 for non-volatile storage.

The data storage 620 may store one or more operating systems (O/S) 622; one or more optional database management systems (DBMS) 624; and one or more program module(s), applications, engines, computer-executable code, scripts, or the like such as, for example, one or more implementation modules 626, communication modules 628, user data modules 629, machine learning modules 630, and/or response modules 631. Some or all of these modules may be sub-modules. Any of the components depicted as being stored in data storage 620 may include any combination of software, firmware, and/or hardware. The software and/or firmware may include computer-executable code, instructions, or the like that may be loaded into the memory 604 for execution by one or more of the processor(s) 602. Any of the components depicted as being stored in data storage 620 may support functionality described in reference to correspondingly named components earlier in this disclosure.

Referring now to other illustrative components depicted as being stored in the data storage 620, the O/S 622 may be loaded from the data storage 620 into the memory 604 and may provide an interface between other application software executing on the server 600 and hardware resources of the server 600. More specifically, the O/S 622 may include a set of computer-executable instructions for managing hardware resources of the server 600 and for providing common services to other application programs (e.g., managing memory allocation among various application programs). In certain example embodiments, the O/S 622 may control execution of the other program module(s) to for content rendering. The O/S 622 may include any operating system now known or which may be developed in the future including, but not limited to, any server operating system, any mainframe operating system, or any other proprietary or non-proprietary operating system.

The optional DBMS 624 may be loaded into the memory 604 and may support functionality for accessing, retrieving, storing, and/or manipulating data stored in the memory 604 and/or data stored in the data storage 620. The DBMS 624 may use any of a variety of database models (e.g., relational model, object model, etc.) and may support any of a variety of query languages. The DBMS 624 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like.

The optional input/output (I/O) interface(s) 606 may facilitate the receipt of input information by the server 600 from one or more I/O devices as well as the output of information from the server 600 to the one or more I/O devices. The I/O devices may include any of a variety of components such as a display or display screen having a touch surface or touchscreen; an audio output device for producing sound, such as a speaker; an audio capture device, such as a microphone; an image and/or video capture device, such as a camera; and so forth. Any of these components may be integrated into the server 600 or may be separate.

The server 600 may further include one or more network interface(s) 608 via which the server 600 may communicate with any of a variety of other systems, platforms, networks, devices, and so forth. The network interface(s) 608 may enable communication, for example, with one or more wireless routers, one or more host servers, one or more web servers, and the like via one or more of networks.

The antenna(s) 634 may include any suitable type of antenna depending, for example, on the communications protocols used to transmit or receive signals via the antenna(s) 634. Non-limiting examples of suitable antennas may include directional antennas, non-directional antennas, dipole antennas, folded dipole antennas, patch antennas, multiple-input multiple-output (MIMO) antennas, or the like. The antenna(s) 634 may be communicatively coupled to one or more transceivers 612 or radio components to which or from which signals may be transmitted or received. Antenna(s) 634 may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals including BLE signals, a Near Field Communication (NFC) antenna for transmitting or receiving NFC signals, a 900 MHz antenna, and so forth.

The transceiver(s) 612 may include any suitable radio component(s) for, in cooperation with the antenna(s) 634, transmitting or receiving radio frequency (RF) signals in the bandwidth and/or channels corresponding to the communications protocols utilized by the server 600 to communicate with other devices. The transceiver(s) 612 may include hardware, software, and/or firmware for modulating, transmitting, or receiving-potentially in cooperation with any of antenna(s) 634—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. The transceiver(s) 612 may further include hardware, firmware, or software for receiving GNSS signals. The transceiver(s) 612 may include any known receiver and baseband suitable for communicating via the communications protocols utilized by the server 600. The transceiver(s) 612 may further include a low noise amplifier (LNA), additional signal amplifiers, an analog-to-digital (A/D) converter, one or more buffers, a digital baseband, or the like.

Referring now to functionality supported by the various program module(s) depicted in FIG. 6, the implementation module(s) 626 may include computer-executable instructions, code, or the like that responsive to execution by one or more of the processor(s) 602 may perform functions including, but not limited to, overseeing coordination and interaction between one or more modules and computer executable instructions in data storage 620, overseeing execution of one or more modules in the health care monitoring system, determining user selected actions and tasks, determining actions associated with user interactions, determining actions associated with user input, initiating commands locally or at remote devices, and the like.

The communication module(s) 628 may include computer-executable instructions, code, or the like that responsive to execution by one or more of the processor(s) 602 may perform functions including, but not limited to, communicating with one or more devices, for example, via wired or wireless communication, communicating with servers (e.g., remote servers), communicating with datastores and/or databases, communicating with user devices and/or user devices, sending or receiving notifications or commands/directives, communicating with cache memory data, communicating with computing devices, and the like.

The user data module(s) 629 may include computer-executable instructions, code, or the like that responsive to execution by one or more of the processor(s) 602 may perform functions including, but not limited to, receiving user data from one or more user devices, receiving survey data from one or more user devices, receiving survey data from healthcare provider devices, and/or receiving any other information relating to the user from the user and/or healthcare provider devices.

The machine learning (ML) module(s) 630 may include computer-executable instructions, code, or the like that responsive to execution by one or more of the processor(s) 602 may perform functions including, but not limited to, running and executing one or more machine learning model such as an embedding, classification, regression RDNN, convolutional neural network (CNN) or any other type of machine learning model.

The response module(s) 631 may include computer-executable instructions, code, or the like that responsive to execution by one or more of the processor(s) 602 may perform functions including, but not limited to, analyzing the output of the machine learning model and generating a response based on the output such as an alert, a report, a recommended action, and/or any other response based on the output of the machine learning model.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, or the like disclosed herein may include one or more software components, including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component including assembly language instructions may require conversion into executable machine code by an assembler prior to execution by the hardware architecture and/or platform.

Another example programming language may be a higher-level programming language that may be portable across multiple architectures. A software component including higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component including instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may include other custom-developed application software, operating system functionality (e.g., device drivers, data storage (e.g., file management) routines, other common routines, and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for monitoring mental health of a user, the method comprising:
training a machine learning algorithm using a plurality of general user data comprising general physiological, general activity, and/or general sleep data corresponding to a plurality of general users different than the user and generate a score indicative of a degree of depression;
receiving a plurality of first user data from at least one user device, the plurality of first user data comprising first physiological, first activity, and/or first sleep data corresponding to the user and associated with a first time period;
receiving survey data corresponding to at least one depressive state of the user and associated with the first time period;
updating the machine learning algorithm by further training the machine learning algorithm using the plurality of first user data and the survey data resulting in an updated machine learning algorithm;
receiving a plurality of second user data from at least one user device, the plurality of second user data corresponding to second physiological, second activity, and/or second sleep data corresponding to the user and associated with a second time period after the first time period;
transforming the plurality of second user data into at least one vector, the at least one vector representative of the plurality of second user data; and
generating a first score using the updated machine learning algorithm and the at least one vector, the first score indicative of a first degree of depression of the user at the second time period.

2. The method of claim 1, wherein the updated machine learning algorithm comprises an input layer, a plurality of feature extraction layers, a plurality of recurrent layers, and at least one dense layer that is a fully connected layer.

3. The method of claim 2, further comprising determining historical data corresponding to the user comprising demographic data, medical history data, clinical data, and/or family history data.

4. The method of claim 3, wherein at least the dense layer processes the historical data and the updated machine learning algorithm generates the first score based on both the historical data and the plurality of second user data.

5. The method of claim 2, wherein the plurality of second user data comprises first data corresponding to a first 24 hour period within the second time period and second data corresponding to a second 24 hour period within the second time period, wherein the plurality of feature extraction layers comprise a first feature extraction layer and a second feature extraction layer, and wherein the first feature extraction layer processes the first data and the second feature extraction layer processes the second data.

6. The method of claim 1, wherein the updated machine learning algorithm is a recurrent deep neural network (RDNN).

7. The method of claim 1, wherein the updated machine learning algorithm comprises a regression model and a classification model.

8. The method of claim 1, further comprising:
comparing the first score to a predefined threshold value corresponding to a depression relapse; and
determining the first score exceeds the predefined threshold value indicating a presence of the depression relapse at the second time period.

9. The method of claim 8, further comprising sending, based on the first score exceeding the predefined threshold value, an alert to a user device and/or a health care provider device corresponding to the presence of the depression relapse at the second time period.

10. The method of claim 9, wherein the alert is sent to the user device, the method further comprising receiving feedback data from the user device after the alert is sent to the user device, the feedback data rejecting the first score and/or the alert.

11. The method of claim 10, further comprising updating the updated machine learning algorithm by further training the updated machine learning algorithm using the feedback data.

12. The method of claim 8, further comprising:
determining, based on the first score exceeding the predefined threshold value, a corrective action corresponding to the presence of the depression relapse; and
sending instructions corresponding to the corrective action to the user device.

13. The method of claim 12, wherein the corrective action corresponds to one of an exercise recommendation, a diet recommendation, a medication recommendation, or a recommendation to meet with a healthcare provider.

14. The method of claim 8, further comprising:
generating, automatically, a report comprising the plurality of second user data and the first score; and
sending the report to a healthcare provider device.

15. The method of claim 1, wherein the plurality of user data comprises heart rate data, electrocardiogram data, sleep data, temperature data, breathing data, step data, exercise data, screen time data, media data, call data, text data, email data, and/or location data.

16. A system for monitoring mental health of a user, the system comprising:
memory configured to store computer-executable instructions; and
at least one computer processor configured to access memory and execute the computer-executable instructions to:
train a machine learning algorithm using a plurality of general user data comprising general physiological, general activity and/or general sleep data corresponding to a plurality of general users different than the user and generate a score indicative of a degree of depression;
receive a plurality of first user data from at least one user device, the plurality of first user data comprising first physiological, first activity, and/or first sleep data corresponding to the user and associated with a first time period;
receive survey data corresponding to at least one depressive state of the user and associated with the first time period;
update the machine learning algorithm by further training the machine learning algorithm using the plurality of first user data and the survey data resulting in an updated machine learning algorithm;
receive a plurality of second user data from at least one user device, the plurality of second user data corresponding to second physiological, second activity, and/or second sleep data corresponding to the user and associated with a second time period after the first time period;
transform the plurality of second user data into at least one vector, the at least one vector representative of the plurality of second user data; and
generate a first score using the updated machine learning algorithm and the at least one vector, the first score indicative of a first degree of depression of the user at the second time period.

17. The system of claim 16, wherein the updated machine learning algorithm comprises an input layer, a plurality of feature extraction layers, a plurality of recurrent layers, and at least one dense layer that is a fully connected layer.

18. The system of claim 17, wherein the at least one computer processor is further configured to access memory and execute the computer-executable instructions to determine historical data corresponding to the user and comprising demographic data, medical history data, clinical data, and/or family history data.

19. The system of claim 18, wherein at least the dense layer processes the historical data and the updated machine learning algorithm generates the first score based on both the historical data and the plurality of second user data.

20. The system of claim 17, wherein the plurality of second user data comprises first data corresponding to a first 24 hour period within the second time period and second data corresponding to a second 24 hour period within the second time period, wherein the plurality of feature extraction layers comprise a first feature extraction layer and a second feature extraction layer, and wherein the first feature extraction layer processes the first data and the second feature extraction layer processes the second data.

21. The system of claim 16, wherein the updated machine learning algorithm is a recurrent deep neural network (RDNN).

22. The system of claim 16, wherein the updated machine learning algorithm comprises a regression model and a classification model.

23. The system of claim 16, wherein the at least one computer processor is further configured to access memory and execute the computer-executable instructions to:
compare the first score to a predefined threshold value corresponding to a depression relapse; and
determine the first score exceeds the predefined threshold value indicating a presence of the depression relapse at the second time period.

24. The system of claim 23, wherein the at least one computer processor is further configured to access memory and execute the computer-executable instructions to send, based on the first score exceeding the predefined threshold value, an alert to a user device and/or a health care provider device corresponding to the presence of the depression relapse at the second time period.

25. The system of claim 24, wherein the alert is sent to the user device and wherein the at least one computer processor is further configured to access memory and execute the computer-executable instructions to receive feedback data from the user device after the alert is sent to the user device, the feedback data rejecting the first score and/or the alert.

26. The system of claim 25, wherein the at least one computer processor is further configured to access memory and execute the computer-executable instructions to update the updated machine learning algorithm by further training the updated machine learning algorithm using the feedback data.

27. The system of claim 23, wherein the at least one computer processor is further configured to access memory and execute the computer-executable instructions to:
determine, based on the first score exceeding the predefined threshold value, a corrective action corresponding to the presence of the depression relapse; and
send instructions corresponding to the corrective action to the user device.

28. The system of claim 27, wherein the corrective action corresponds to one of an exercise recommendation, a diet recommendation, a medication recommendation, or a recommendation to meet with a healthcare provider.

29. The system of claim 23, wherein the at least one computer processor is further configured to access memory and execute the computer-executable instructions to:
generate, automatically, a report comprising the plurality of second user data and the first score; and
send the report to a healthcare provider device.

30. The system of claim 16, wherein the plurality of user data comprises heart rate data, electrocardiogram data, sleep data, temperature data, breathing data, step data, exercise data, screen time data, media data, call data, text data, email data, and/or location data.
